(19) United States
(12) Patent Application Publication
(10) Pub. No.: US 2004/0266827 A1
(43) Pub. Date: Dec. 30, 2004

(54) Convergent asymmetric route to produce a key intermediate towards the synthesis of a garft inhibitor

(75) Inventors: Wolfgang Reinhard Ludwig Notz, La Jolla, CA (US); Carlos Alberto Martinez, Oceanside, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 10/870,311

(22) Filed: Jun. 16, 2004

Related U.S. Application Data
(60) Provisional application No. 60/482,321, filed on Jun. 25, 2003.

(51) Int. Cl. C 07D 4 9/02

(52) U.S. Cl. 514/326; 546/207

(57) ABSTRACT

The invention relates to processes for the preparation of a key intermediate in the synthesis of a GARFT inhibitor of formula (Ia) containing a 4-methyl-substituted thiophene core:

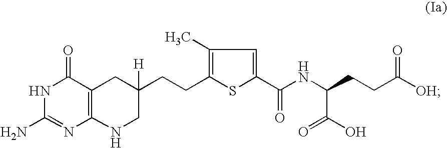

wherein said key intermediate has the following formula (I):

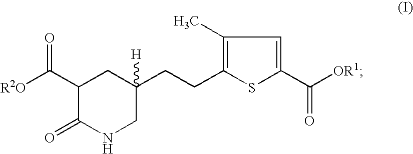

wherein each of R¹ and R² are independently a moiety that together with the attached CO₂ forms a readily hydrolyzable ester group;

from a compound of the formula (VI):

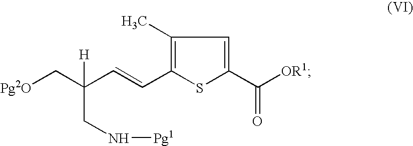

wherein R¹ is as described above, Pg¹ is an amino protecting group, and Pg² is an ether protecting group; wherein said processes are as described in the specification.

This application claims the benefit of U.S. Provisional Application No. 60/482,321 filed on Jun. 25, 2003, the contents of which is hereby incorporated by reference in its entirety.

### BACKGROUND OF THE INVENTION

The present invention relates to a new stereoselective method for the preparation of a key intermediate towards the synthesis of a GARFT inhibitor containing a methyl-substituted thiophene core.

The large class of antiproliferative agents includes antimetabolite compounds. A particular subclass of antimetabolites known as antifolates or antifoles are antagonists of the vitamin folic acid. Typically, antifolates closely resemble the structure of folic acid and incorporate the characteristic P-benzoyl glutamate moiety of folic acid. The glutamate moiety of folic acid takes on a double negative charge at physiological pH. Therefore, this compound and its analogs have an active energy driven transport system to cross the cell membrane and exert a metabolic effect. Glycinamide ribonucleotide formyl transferase (GARFT) is a folate dependent enzyme in the de novo purine biosynthesis pathway. This pathway is critical to cell division and proliferation. Shutting down this pathway is known to have an antiproliferative effect, in particular, an antitumor effect. Thus, a number of folate analogs have been synthesized and studied for their ability to inhibit GARFT. A prototypical specific tight binding inhibitor of GARFT, 5,10-dideazatetrahydrofolic acid, has been reported to show antitumor activity. See F. M. Muggia, “Folate antimetabolites inhibitor to de novo purine synthesis,” New Drugs, Concepts and Results in Cancer Chemotherapy, Kluwer Academic Publishers, Boston (1992), 65-87. One such GARFT inhibitor is:

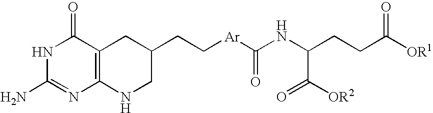

wherein Ar is a substituted or unsubstituted five- or six-membered aromatic group, and wherein each of R¹ and R² are independently a hydrogen atom or a moiety that together with the attached CO₂ forms a readily hydrolyzable ester group. Compounds of such formula are generically described in U.S. Pat. No. 5,646,141, the disclosure of which is incorporated by reference herein. One process of preparing such GARFT inhibitors is described in U.S. Pat. No. 5,981,748, the disclosure of which is incorporated by reference herein. Other patent directed to GARFT inhibitors includes U.S. Pat. No. 5,608,082, the disclosure of which is incorporated by reference herein.

An efficient preparation of a GARFT inhibitor wherein Ar is 4-methyl-thien-2,5-diyl is desirable.

### SUMMARY OF THE INVENTION

This invention is directed to processes for the preparation of a key intermediate in the synthesis of a GARFT inhibitor of formula (Ia) containing a 4-methyl-substituted thiophene core:

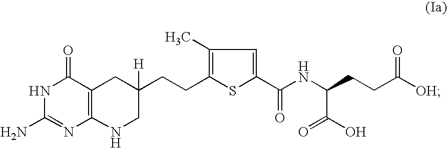

wherein said intermediate has the following formula (1):

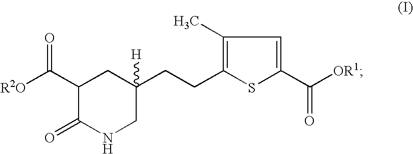

wherein each of R¹ and R² are independently a moiety that together with the attached CO₂ forms a readily hydrolyzable ester group;

from a compound of formula (VIa):

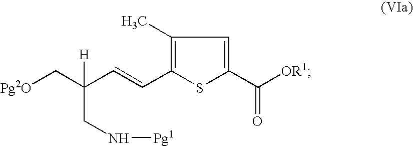

wherein R¹ is as described above, Pg¹ is an amino protecting group, Pg² is an ether protecting group; for the preparation of a compound or a salt of formula (Ia):

Suitably, each R¹ and R² are independently C₁-C₆ alkyl, —(CR¹³R¹⁴)ₜ(C₆-C₁₀ aryl) and —(CR¹³R¹⁴)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl. Preferably, each R¹ and R² are independently C₁-C₆ alkyl or benzyl. More preferably, each R¹ and R² are independently methyl or ethyl.

Preferably Pg¹ is an acyl, more preferably benzyloxycarbonyl.

Preferably Pg² is a silyl ether protecting group, more preferably tert-butyidimethylsilyl.

In a specific embodiment, this invention relates to a method of preparing a compound or a salt of formula (I):

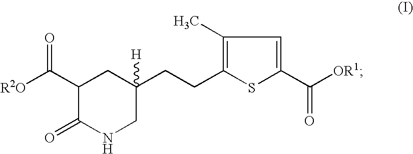

wherein each of R¹ and R² are as described above;

wherein the method comprises the following steps:

(a) reacting a compound of formula (VI) selected from the group consisting of:

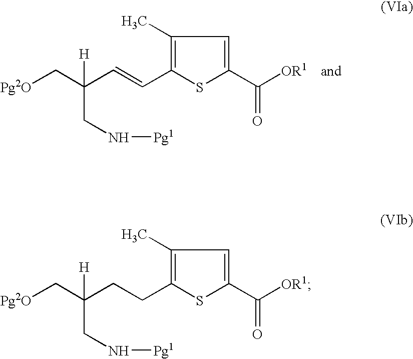

wherein R¹ is as described above, Pg¹ is an amino protecting group, and Pg² is an ether protecting group; with a deprotecting agent in a solvent, to form a compound of formula (V) selected from the group consisting of:

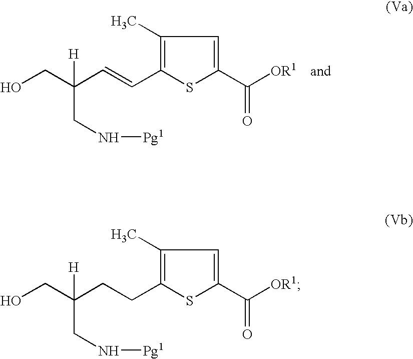

wherein each of Pg¹ and R¹ are as described above;

(b) reacting said compound of formula (V) with a leaving group producing agent in the presence of a base in a solvent, to form a compound of formula (IV) selected from the group consisting of:

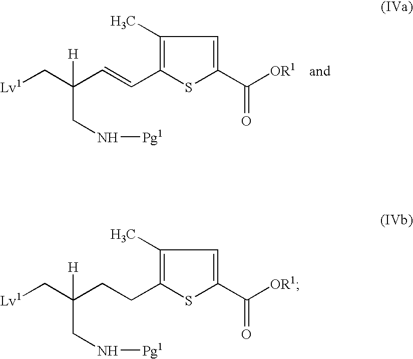

wherein each of Pg¹ and R¹ are as described above, and Lv¹ is a leaving group;

(c) reacting said compound of formula (IV) with a compound of formula (III):

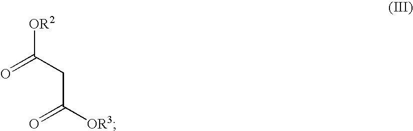

wherein R² is as described above, and R³ is a moiety that together with the attached CO₂ forms a readily hydrolyzable ester group; in the presence of a base in a solvent, to form a compound of formula (II) selected from the group consisting of:

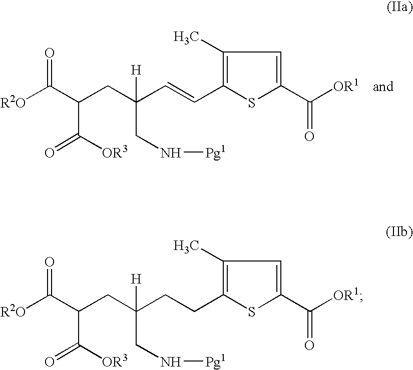

wherein each of Pg¹, R¹, R², and R³ are as described above; and

(d) reacting said compound of formula (II) with a cyclization reagent to form said compound of formula (I);

wherein the following hydrogenation step (e) is performed before any one of the above step (a), step (b), step (c), or step (d):

(e) hydrogenating the double bond in said compound (VIa), (Va), (IVa), or (IIa) by reacting said compound of formula (VIa), (Va), (IVa), or (IIa) with a hydrogenating agent in the presence of a catalyst and a solvent, to form a compound of formula (VIb), (Vb), (IVb), or (IIb); respectively.

Suitably, R³ is C₁-C₆ alkyl, —(CR¹³R¹⁴)ₜ(C₆-C₁₀ aryl) or —(CR¹³R¹⁴)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl. Preferably, R³ is independently C₁-C₆ alkyl or benzyl. More preferably, R³ is methyl or ethyl.

In step (a), preferably said deprotecting agent is tetrabutyl ammonium fluoride. Preferably said solvent is tetrahydrofuran.

In step (b), preferably said leaving group producing agent is mesyl chloride. Preferably said base is an amine base, more preferably triethylamine. Preferably said solvent is a polar aprotic solvent, more preferably methylene chloride.

In step (c), preferably said base is hydride or (C₁-C₈)alkoxide, more preferably sodium hydride. Preferably step (c) is performed in the presence of a catalyst, preferably halide salt, more preferably sodium iodide, at an elevated temperature, preferably at a reflux temperature. Preferably said solvent is a polar aprotic solvent, more preferably tetrahydrofuran.

In step (d), suitably said cyclizabon reagent is an acid, preferably a mixture of hydrobromic acid and acetic acid, followed by treatment with a base, preferably potassium hydroxide.

In step (e), suitably said hydrogenating agent is any known hydrogenating agents, preferably hydrogen. Suitably the catalyst is a metal catalyst, preferably palladium on carbon or palladium hydroxide on carbon. Preferably the solvent is an alcoholic solvent, more preferably methanol.

In one embodiment, the hydrogenation step (e) is perfomed before the above step (a) in the following manner:

(e) hydrogenating the double bond in compound (VIa) by reacting said compound of formula (VIa) with a hydrogenating agent in the presence of a catalyst and a solvent, to form a compound of formula (VIb):

(a) reacting said compound of formula (VIb) with a deprotecting group in a solvent, to form said compound of formula (Vb);

(b) reacting said compound of formula (Vb) with a leaving group producing agent in the presence of a base in a solvent, to form said compound of formula (IVb);

(c) reacting said compound of formula (IVb) with said compound of formula (III) in the presence of a base in a solvent, to form said compound of formula (IIb); and

(d) reacting said compound of formula (IIb) with a cyclization reagent to form said compound of formula (I).

In another embodiment, the hydrogenation step (e) is perfomed before the above step (b) in the following manner:

(a) reacting said compound of formula (VIa) with a deprotecting group in a solvent, to form said compound of formula (Va);

(e) hydrogenating the double bond in compound (Va) by reacting said compound of formula (Va) with a hydrogenating agent in the presence of a catalyst and a solvent, to form said compound of formula (Vb);

(b) reacting said compound of formula (Vb) with a leaving group producing agent in the presence of a base in a solvent, to form said compound of formula (IVb);

(c) reacting said compound of formula (IVb) with said compound of formula (III) in the presence of a base in a solvent, to form said compound of formula (IIb); and

(d) reacting said compound of formula (IIb) with a cyclization reagent to form said compound of formula (I).

In yet another embodiment, the hydrogenation step (e) is perfomed before the above step (c) in the following manner:

(a) reacting said compound of formula (VIa) with a deprotecting group in a solvent, to form said compound of formula (Va);

(b) reacting said compound of formula (Va) with a leaving group producing agent in the presence of a base in a solvent, to form said compound of formula (IVa);

(e) hydrogenating the double bond in said compound (IVa) with a hydrogenating agent in the presence of a catalyst and a solvent, to form said compound of formula (IVb);

(c) reacting said compound of formula (IVb) with said compound of formula (III) in the presence of a base in a solvent, to form said compound of formula (IIb); and

(d) reacting said compound of formula (IIb) with a cyclization reagent to form said compound of formula (I).

In a preferred embodiment, the hydrogenation step (e) is perfomed before the above step (d) in the following manner:

(a) reacting said compound of formula (VIa) with a deprotecting group in a solvent, to form said compound of formula (Va);

(b) reacting said compound of formula (Va) with a leaving group producing agent in the presence of a base in a solvent, to form said compound of formula (IVa);

(c) reacting said compound of formula (IVa) with said compound of formula (III) in the presence of a base in a solvent, to form said compound of formula (IIa);

(e) hydrogenating the double bond in said compound (IIa) by reacting said compound of formula (IIa) with a hydrogenating agent in the presence of a catalyst and a solvent, to form said compound of formula (IIb); and

(d) reacting said compound of formula (IIb) with a cyclization reagent to form said compound of formula (I).

Within this preferred embodiment, in said step (d), said cyclisizing reagent is an acid in a solvent followed by a basic work-up. Preferably said acid is HBr. Preferably said solvent is a polar protic solvent. Preferably said basic workup is aqueous base, more preferably aqueous bicarbonate, hydroxide, or mixtures thereof, most preferably aqueous NaHCO₃ and KOH.

Within this preferred embodiment, in said step (d), the following intermediate of formula (VII) is formed:

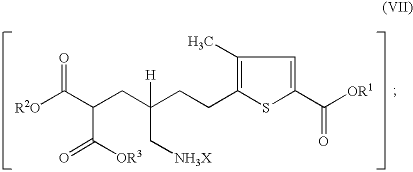

wherein R¹, R², and R³ are as described above, and X is halo, preferably bromo.

Each of R¹, R², and R³ are independently C₁-C₆ alkyl, —(CR¹³R¹⁴)ₜ(C₆-C₁₀ aryl) and —(CR¹³R¹⁴)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl.

In another embodiment, the method further comprises the following step of preparing said compound of formula (VIa):

(f-1) reacting a compound of formula (VIIIa):

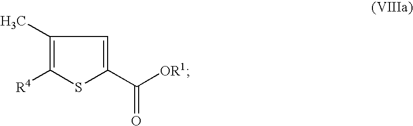

wherein R¹ is as described above and R⁴ is selected from the group consisting of —(CH₂PO(OR⁶)₂), —(CH₂P(R⁷)₃)⁺X⁻, —CH═P(R⁷)₃, —CH₂MX, —CH₂Si(R⁷)₃, and —CH₂SO₂(R⁷);

wherein each R⁶ and R⁷ is independently C₁-C₆ alkyl, —(CR¹³R¹⁴)ₜ(C₆-C₁₀ aryl) and —(CR¹³R¹⁴)ₜ (4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl;

X is halo; and M is a metal;

with a compound of formula (IXa):

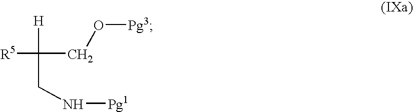

wherein Pg¹ is as described above, Pg³ is an ether protecting group, and R⁵ is —(C═O)—H;

in the presence of a base, to form said compound of formula (VI).

Within this embodiment, preferably said R⁴ is —(CH₂P(R⁷)₃)⁺X⁻, wherein R⁷ is C₁-C₆ alkyl, or benzyl. More preferably R⁴ is —(CH₂P(CH₂CH₃)₃)⁺Br⁻.

Preferably M is magnesium, zinc, or copper.

Preferably R⁶ is —(CR¹³R¹⁴)ₜ(C₆-C₁₀ aryl), wherein t is 0 or 1 and R¹³ and R¹⁴ is independently H.

Within this embodiment, preferably R⁷ is —(CR¹³R¹⁴)ₜ(C₆-C₁₀ aryl), wherein t is 0 or 1 and R¹³ and R¹⁴ is independently H.

In yet another embodiment, the method further comprises the following steps of preparing said compound of formula (VIa):

(f-2) reacting a compound of formula (VIIIb):

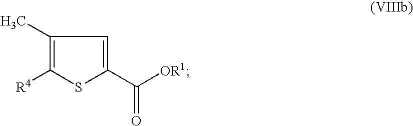

wherein R¹ is as described above and R⁴ is —(C═O)—H;

with a compound of formula (IXb):

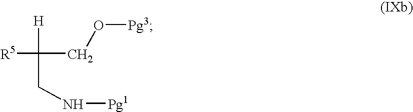

wherein Pg¹ is as described above, Pg³ is an ether protecting group, and R⁵ is selected from the group consisting of —(CH₂PO(OR⁶)₂), —(CH₂P(R⁷)₃)⁺X⁻, —CH═P(R⁷)₃, —CH₂MX, —CH₂Si(R⁷)₃, and —CH₂SO₂(R⁷), wherein each R⁶ and R⁷ are as described above; in the presence of a base in an inert solvent, to form said compound of formula (VI).

Preferably Pg³ is tert-butyldimethylsilyl.

Suitably the bases employed in step (f-2) include (C₁-C₈)alkoxide, hydride, LHMDS, DBU and lithium halide, (C₁-C₈)alkyl lithium, or (C₆-C₁₀)aryl lithium. Preferably, the bases are selected from the group consisting of (C₁-C₈)alkoxide and hydride.

Within this embodiment, suitably the inert solvents include hydrocarbons and ethers, preferably tetrahydrofuran.

Within this embodiment, X is halo, preferably bromo or chloro.

In another embodiment, the method further comprises the following steps of preparing said compound of formula (VIIIa):

(g) reacting a compound of formula (X):

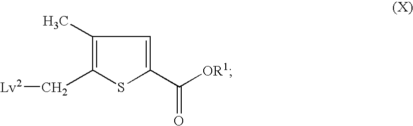

with an activating agent, wherein R¹ is as described above and Lv² is a leaving group, in a solvent, to form said compound of formula (VIIIa).

Suitable activating agents are those known to those skilled in the art, including those compounds which produce R in said compound of the formula (Villa) as follows: when R⁴ is —(CH₂PO(OR⁶)₂), the suitable activating agents include P(OR)₃ or HPO(OR⁶)₂; when R⁴ is —(CH₂P(R⁷)₃)⁺X⁻ or —CH═P(R⁷)₃, the suitable activating agents include P(R⁷)₃; when R⁴ is —CH₂MX, the suitable activating agents include metal, such as Mg or Zn; when R⁴ is CH₂Si(R⁷)₃, the suitable activating agents include metal, to first form the —CH₂MX intermediate, followed by XSi(R⁷)₃; when R⁴ is CH₂SO₂(R⁷), the suitable activating agents include SR⁷, followed by oxidation to sulfone by methods known to those skilled in the art. Each R⁶ and R⁷ are as described above.

Preferably said activating agent is P(R⁷)₃, wherein R⁷ is (C₁-C₈)alkyl, more preferably wherein R⁷ is ethyl. Preferably said solvent is toluene.

In another embodiment, the method further comprises the following steps of preparing said compound of formula (IXa):

(h) reacting a compound of formula (XI):

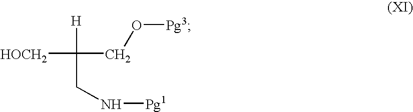

wherein Pg¹ and Pg³ are as described above, with an oxidizing agent in a suitable solvent, to form said compound of formula (IXa).

Suitably, the oxidizing agents include any oxidizing agents including methylsulfoxide-based Swern-type oxidizing agents, chromium-based oxidizing agents, and iodine-based oxidizing agents. Preferably the oxidizing agent is Dess-Martin periodinane or a Swem-type oxidizing agent such as pyridine-SO₃ complex in the presence of Hunigs base.

Suitably, the solvents include hydrocarbons and ethers, preferably dichloromethane or tetrahydrofuran.

In another embodiment, the method further comprises the following steps of preparing said compound of formula (XI):

(i-1) reacting a compound of formula (XII):

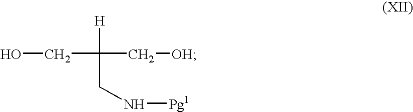

wherein Pg¹ is as described above;

with a compound of formula R⁹—(O═C)—O—(C═O)—R⁹; wherein R⁹ is selected from the group consisting of C₁-C₆ alkyl, —(CR¹³R¹⁴)ₜ(C₆-C₁₀ aryl) and —(CR¹³R¹⁴)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl, to form a compound of formula (XIII):

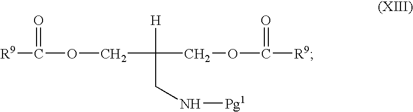

wherein Pg¹ and R⁹ are independently as described above;

(j-1) exposing said compound (XIII), to an enzyme, in the presence of a buffer, to form a compound of formula (XIV):

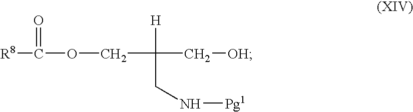

(j-2) reacting said compound of formula (XIV) with a Pg³ producing agent in the presence of a base, followed by base-induced cleavage of R⁸—(C═O)— to form said compound of formula (XI).

Within this embodiment, preferably each of said R⁸ and R⁹ is independently C₁-C₆ alkyl or benzyl, more preferably methyl.

Within this embodiment, preferably said enzyme is P. cepacia lipase.

Within this embodiment, suitably in the step (j-1), the method is stereoselective and provides an enantiomeric excess of at least 60% to 99.9%, preferably at least 80% to 99%.

In another embodiment, the method further comprises the following steps of preparing said compound of formula (XI):

(i-2) exposing a compound of formula (XII):

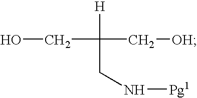

wherein Pg¹ is as described above;

to an enzyme, in the presence of a compound of formula CH₂═CH—O—(C═O)—R⁸, to form a compound of formula (XIV):

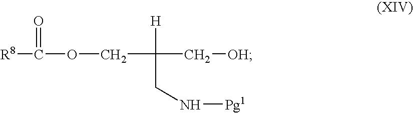

(j-2) reacting said compound of formula (XIV) with a Pg³ producing agent in the presence of a base, followed by base-induced cleavage of R⁸—(C═O)— to form said compound of formula (XI).

Within this embodiment, preferably said R⁸ is CH₃, said enzyme is P. cepacia lipase, and said step (i-2) is performed in the presence of a compound of formula CH₂═CH—O—(C═O)—CH₃ in an alcoholic solvent.

Within this embodiment, suitably in the step (i-2), the method is stereoselective and provides an enantiomeric excess of compound (XI) of at least 60% to 99.9%, preferably at least 80% to 99%.

In another embodiment, the method comprises a further purification step, including derivatizaton, to provide enantiomerically pure compound (XI), i.e., compound (XIa) or (XIb) as described below.

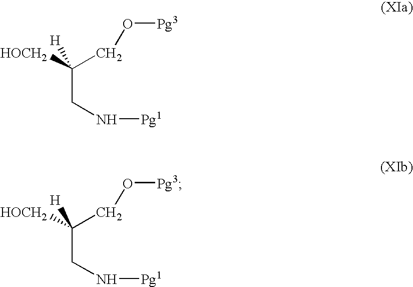

wherein Pg¹ and Pg³ are as described above.

In another embodiment, the method further comprises the following steps of preparing said compound of formula (XI):

(i-3) reacting a compound of formula (XII):

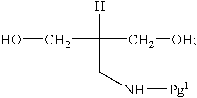

wherein Pg¹ is as described above;

with a Pg³ producing agent in the presence of a base, to form said compound of formula (XI).

In another sub-embodiment of any one of the above embodiments, the method further comprises the following steps of preparing said compound of formula (XII):

(k) reacting a compound of formula (XV):

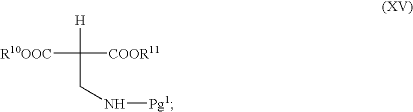

wherein each of R¹⁰ and R¹¹ are independently a moiety that together with the attached CO₂ forms a readily hydrolyzable ester group and Pg¹ is as described above;

with a reducing agent, to form said compound of formula (XII).

R¹⁰ and R¹¹ are independently selected from the group consisting of C₁-C₆ alkyl, —(CR¹³R¹⁴)ₜ(C₆-C₁₀ aryl) and —(CR¹³R¹⁴R)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl. Preferably, R¹⁰ and R¹¹ are independently C₁-C₆ alkyl or benzyl, more preferably tert-butyl or ethyl.

Suitably, the reducing agents can be any known reducing agents, including silanes, such as polymethylhydrosiloxane (PMHS), trichlorosilane, hexachlorodisilazane, or phenyltrisilane, optionally in the presence of catalysts which comprise monomeric zinc compounds, complexed by basic ligands such as amines, polyamines, aminoalcohols, amine oxides, amides, phosphoramides, etc. The reducing agents can also be a hydride such as LiAlH₄, NaBH₄, or LiBH₄.The reducing agents can also be hydrogen gas in the present of a metal catalyst. Preferably the reducing agent is LiBH₄.

In another sub-embodiment of any one of the above embodiments, the method further comprises the following steps of preparing said compound of formula (XV):

(I) reacting a compound of formula (XVIII):

R¹⁵—O—(C═O)—NH₂ (XVIII);

wherein R¹ is C₁-C₆ alkyl, —(CR¹³R¹⁴)ₜ(C₆-C₁₀ aryl) and —(CR¹³R¹⁴)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl;

with formaldehyde in the presence of a base and a solvent, to form a compound of formula (XVII):

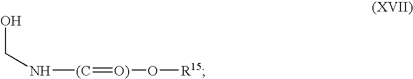

wherein R¹⁵ is as described above;

(m) reacting said compound of formula (XVII) with a compound of formula R¹²—(O═C)—O—(C═O)—R¹², wherein R¹² is C₁-C₆ alkyl, —(CR¹³R¹⁴)ₜ(C₆-C₁₀ aryl) and —(CR¹³R¹⁴)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl; to form a compound of formula (XVI):

Pg¹—NH—CH₂—O—(C═O)—R¹² (XVI);

wherein Pg¹ and R¹² are independently as described above;

(n) reacting said compound of formula (XVI) with a compound of formula R¹¹—O—(O═C)—CH₂—(C═O)—O—R¹⁰ in the presence of a base, wherein each of R¹⁰ and R¹¹ are independently a moiety that together with the attached CO₂ forms a readily hydrolyzable ester group, in the presence of a base and solvent to form said compound of formula (XV).

Preferably R¹² is C₁-C₆ alkyl or benzyl, more preferably methyl or ethyl.

Preferably R¹⁵ is benzyl.

The invention also relates to a compound which is:

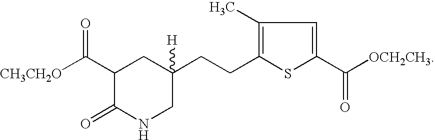

The invention further relates to a compound of formula (V):

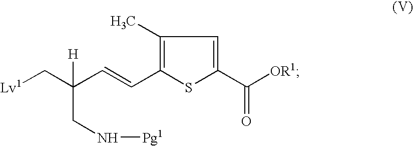

wherein Pg¹ is an amino protecting group, R¹ is a moiety that together with the attached CO₂ forms a readily hydrolyzable ester group, and Lv¹ is a leaving group.

For purposes of the present invention, as described and claimed herein, the following terms are defined as follows:

The term “halo”, as used herein, unless otherwise indicated, means fluoro, chloro, bromo or iodo. Preferred halo groups are fluoro, chloro and bromo.

The term “alkyl”, as used herein, unless otherwise indicated, includes saturated monovalent hydrocarbon radicals having straight or branched moieties.

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of 4-10 membered heterocyclic are derived from, but not limited to, the following:

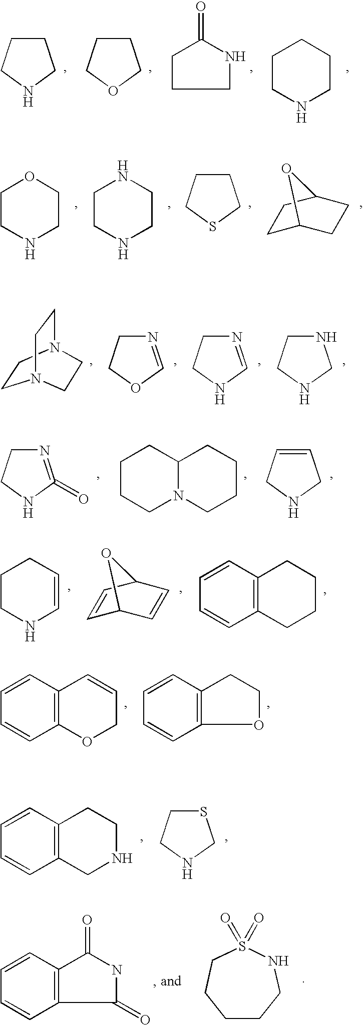

Unless otherwise indicated, the term ‘oxo’ refers to ═O.

Certain compounds of formula (I) may have asymmetric centers and therefore exist in different enantiomeric forms. All optical isomers and stereoisomers of the compounds of formula (I), and mixtures thereof, are considered to be within the scope of the invention. With respect to the compounds of formula (I), the invention includes the use of a racemate, one or more enantiomeric forms, one or more diastereomeric forms, or mixtures thereof. The compounds of formula (I) may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patni et al., Chem. Rev, 1996, 96, 3147-3176 and references cited therein.

The subject invention also includes isotopically-labelled compounds, which are identical to those recited in Formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula (I) of this invention and prodrugs thereof can generally be prepared by carrying out the procedures disclosed in the Schemes and/or in the Examples and Preparations below, by substituting a readily available isotopically labelled reagent for a non-isotopically labelled reagent.

Other aspects, advantages, and preferred features of the invention will become apparent from the detailed description below.

### DETAILED DESCRIPTION OF THE INVENTION AND ITS PREFERRED EMBODIMENTS

According to a process of this invention, compound of formula (I) can be prepared by the reaction schemes depicted below:

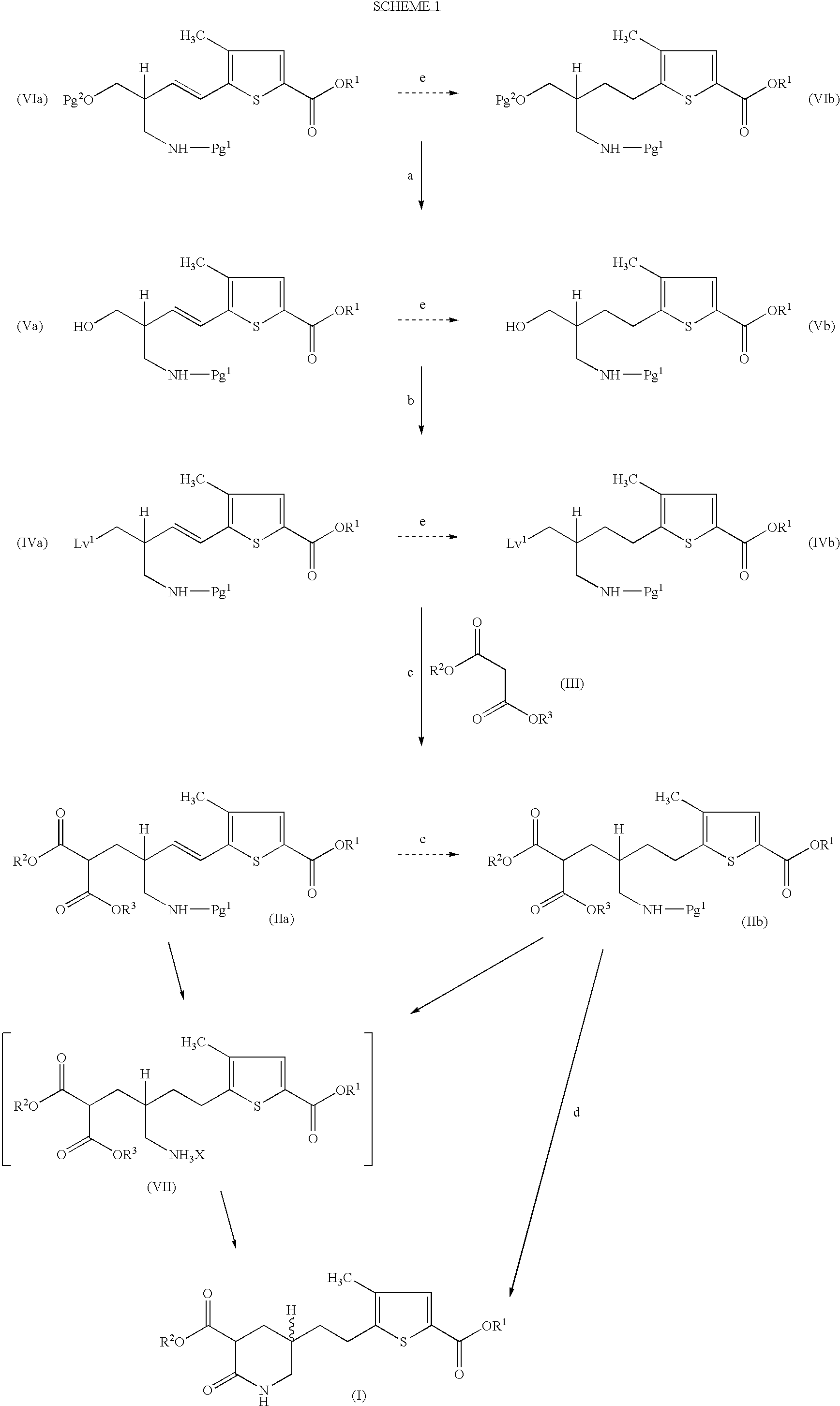

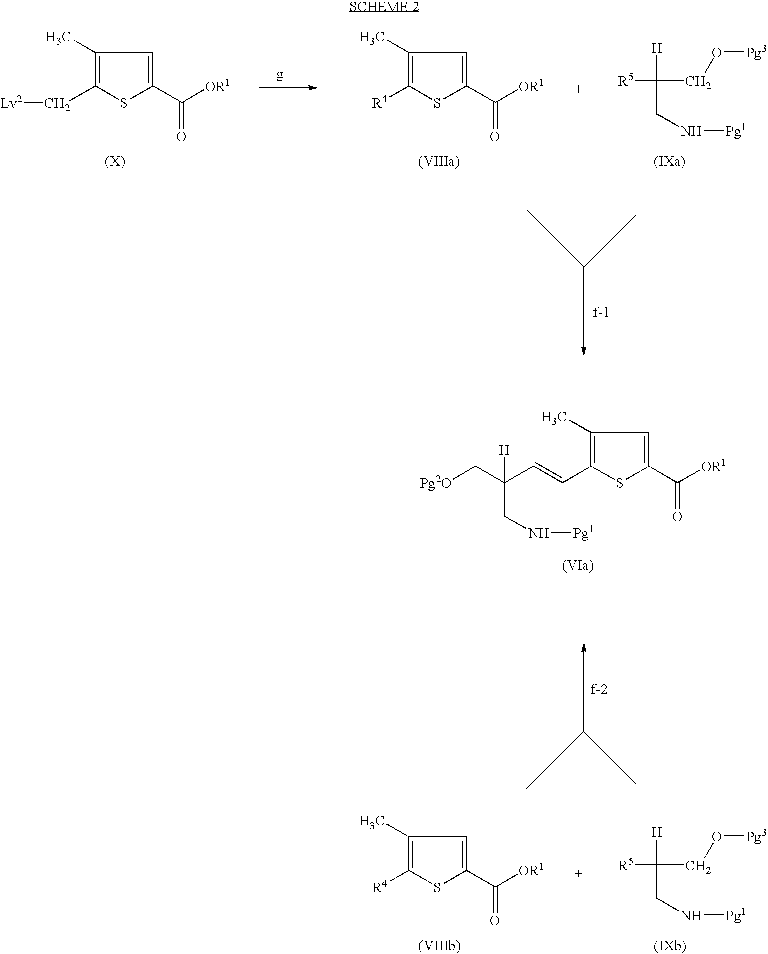

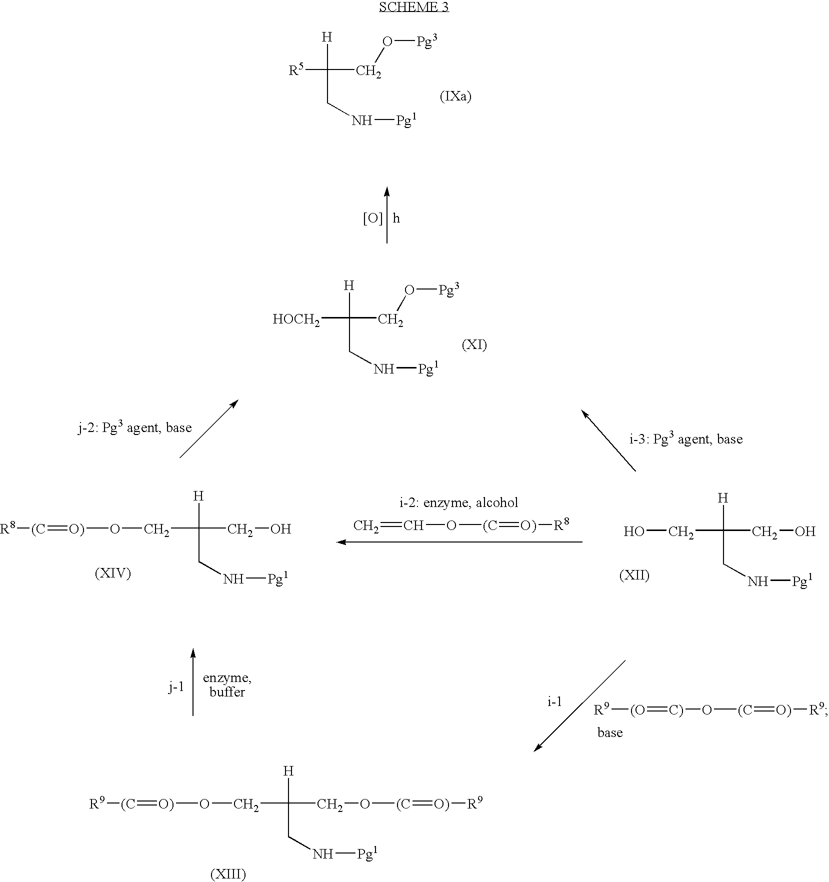

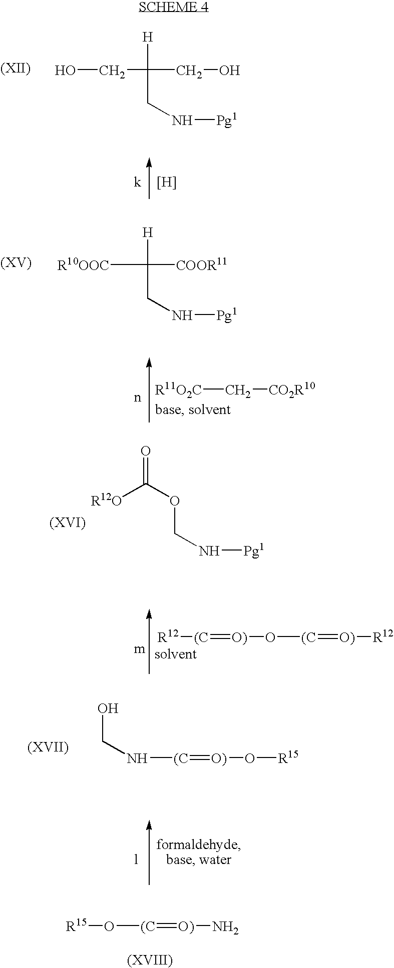

Scheme 1 illustrates the preparation of compounds of formula (I). According to Scheme 1, a compound of formula (I) can generally be prepared as follows from a compound of formula (VIa). Compound of formula (VIa) can be deprotected to remove the protecting group Pg² affording alcohol (Va). The hydroxy functionality of said alcohol (Va) can be to afford compound (IVa). Compound (IVa) can then be reacted with the monoanion of a malonate diester (III) in the presence of a catalyst to form compound (IIa). The double bond of compound (IIa) can then be reduced according to step (e) by catalytic hydrogenation under an atmosphere of hydrogen to form compound (IIb). The Pg¹-group of compound (IIb) can then be removed by treatment with a suitable deprotection agent affording the ammonium salt compound (VII). Compound (VII) was then cyclized in situ by treatment with aqueous base the compound of formula (I).

Scheme 2 illustrates the preparation of compounds of formula (VIa), which can be used as a starting material in Scheme 1. According to Scheme 2, a compound of formula (VIa) can generally be prepared as follows by reacting a compound of formula (VIII), i.e., compound of formula (VIIIa) or (VIIIb), respectively, with a compound of formula (IX), i.e., compound of formula (IXa) or (IXb), respectively. An-ylide or anion, respectively, generated from a compound of formula (VIIIa) can be reacted with a compound of formula (IXa), which results in the formation of Wittig-product (VIa) with trans-configurated double bond. Alternatvely, compound (VIIIb) can be reacted with an ylide or anion, respectively, of a compound of formula (IXb) to form a compound of formula (VIa). Compound of formula (VIIIa) can be prepared by reacting a compound of formula (X) with an activating group, which is capable of acidifying the adjacent methylene group as well as of stabilizing the corresponding anion.

Scheme 3 illustrates the preparation of compounds of formula (IXa), which can be used as a starting material in Scheme 2. According to Scheme 3, in step (i-1), a compound of formula (IXa) can generally be prepared as follows by reacting a compound of formula (XII) with a carboxylic acid anhydride to form a prochiral diester of formula (XIII). Said compound (XIII) can then be desymmetrized by an enzyme to afford compound (XIV) in highly optically pure form. Alternatively, in step (i-2), a compound of formula (IXa) can generally be prepared as follows by reacting a compound of formula (XII), which was enzymatically desymmetrized by enantoselective acylation to afford compound (XIV) with excellent enatiomeric excess. Protection of the alcohol functionality of compound of formula (XIV) followed by deacylation affords said compound of formula (XI) in nearly quanutauve yield. Said compound (XI) can then be oxidized to provide said compound of formula (IXa). Yet alternatively, in step (i-3), said compound of formula (IXa) can also be prepared in a racemic form from a compound of formula (XII) by monoprotecbon of said compound of formula (XII).

Scheme 4 illustrates the preparation of compounds of formula (XII), which can be used as a starting material in Scheme 3. According to Scheme 4, in step (I), a suitable carbamate compound of formula (XVIII) can be hydroxymethylated with aqueous formaldehyde in the presence of a base to form a compound of formula (XVII). In step (m), said compound (XVII) can be acylated by reaction with carboxylic acid anhydride in the presence of a base to afford an aminomethylation agent of formula (XVI), which can then be reacted with an anion of a malonate diester to furnish an aminomethylated compound of formula (XV). Reduction of the ester groups of compound of formula (XV) provides the prochiral diol of formula (XII).

Compounds of formula (XVI) can be prepared as described above or by the methods described in MacDonald et. al., Journal of Medicinal Chemistry 1980, 23(4), 413-420.

Compounds of formula (XV) can be prepared as described above or by the methods described in Katritzky et. al., Journal of Organic Chemistry 2002, 67(14), 4957-4959.

Compounds of the formula (X) are known in the art and may be prepared as described herein or according to the methods described in Choong, Ingrid; Burdeft, Matthew; Delano, Warren; Erlanson, Daniel A. Lee, Dennis; Lew, Willard. (Sunesis Pharmaceuticals, Inc., USA). PCT Int. Appl. (2003), PCT Int. Appl., WO03024955, and WO 0208220, herein incorporated in their entirety by reference.

### EXAMPLES

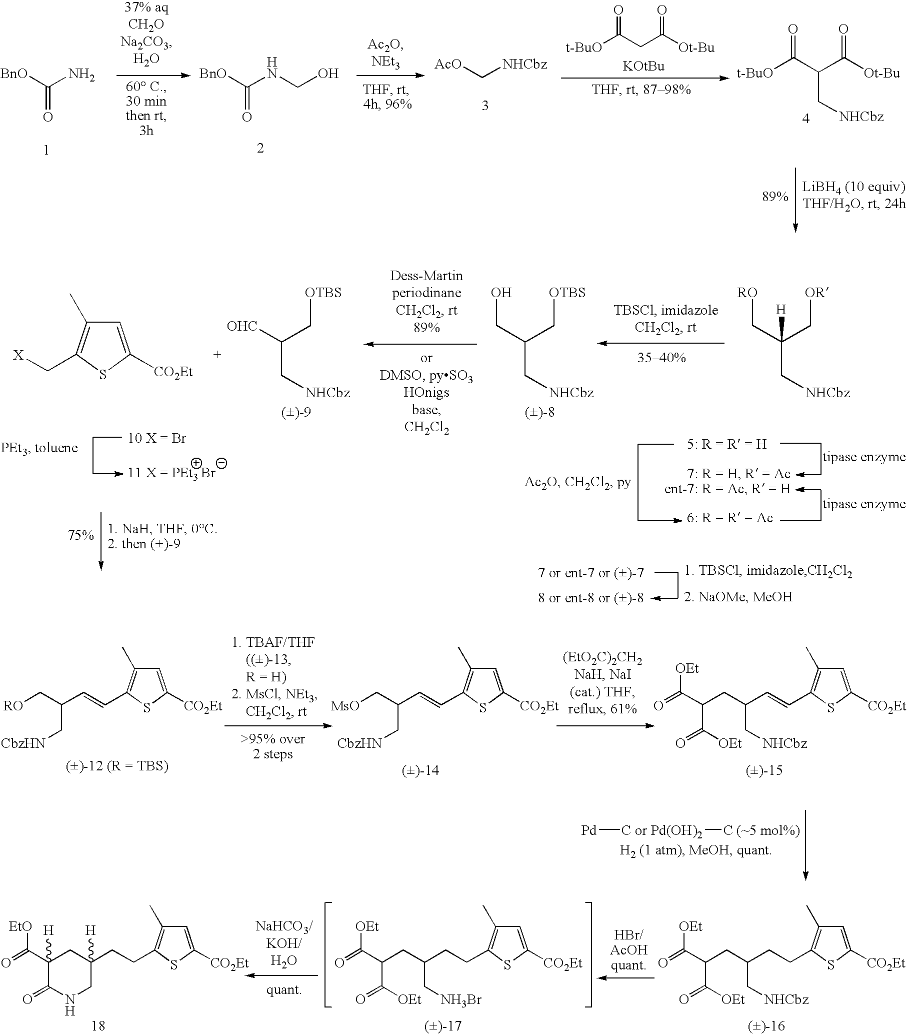

A solution of di-O-tBu-malonate (225 μL; 1.00 mmol) in anhydrous THF (6 mL) is treated with solid KOtBu (110 mg; 0.98 mmol) at room temperature. After 20 min, the resulting malonate anion suspension is then quickly added to a vigorously stirred solution of benzyl N-(acetoxymethylene)carbamate 3 (200 mg; 0.90 mmol) in THF (4 mL) at room temperature. After 1 min, the reaction mixture is quenched by addition of saturated NH₄Cl/H₂O/EtOAc (6 mL, 2:2:2) while stirring vigorously. The two layers are separated, and the aqueous layer is extracted with EtOAc (2×). The combined organic layers are dried (MgSO₄), filtered, concentrated, and purified by flash column chromatography (silica; Hexanes/EtOAc=5:1) to afford aminomethylated product 4 (330 mg; 87%) as a colorless syrup. The analytical data are in accordance with those reported earlier (see Katritzky, Alan R.; Kirichenko, Kostyantyn; Elsayed, Ahamad M.; Ji, Yu; Fang, Yunfeng; Steel, Peter J. Journal of Organic Chemistry 2002, 67,4957-4959). 1H NMR (300 MHz, CDCl₃): 1.36 (s, 18H, 2×tBu); 3.36 (m, 1H, CH); 3.54 (m, 2H, CH₂N); 5.00 (s, 2H, CH₂Ph);5.33 (m, 1H, NH); 7.19-7.26 (m, 5H, Ph). 13C NMR (CDCl₃): 28.145, 28.219, 28.287, 28.357, 40.180, 54.151, 67.059, 82.419, 128.351, 128.793, 137.046, 156.478, 167.690.

Compound of the formula 3 may be prepared by methods known to those skilled in the art. For example, see: MacDonald, Scott A.; Willson, C. Grant; Chorev, Michael; Vernacchia, Fred S.; Goodman, Murray. Journal of Medicinal Chemistry 1980, 23, 413-420.

Reduction of 4 to Prochiral Diol 5

To a solution of 4 (294 mg; 0.775 mmol) in THF (8 mL) is added H₂O (68 μL), followed by LiBH₄ (2 mL; 2M solution in THF) at 0° C. After the addition of the LiBH4 solution is complete, the reaction mixture is stirred for 4-5 h at room temperature. Then, upon cooling with an ice-bath, the reaction is carefully quenched by addition of aqueous 3N HCl until the excess of LiBH₄ has been destroyed resulting in a clear solution. Then, half-staturated aqueous NaCl-solution is added, and the mixture is extracted with EtOAc. The combined EtOAc-phases are dried (MgSO₄), filtered, concentrated, and purified by flash column chromatography (silica; Hexanes/EtOAc=1:1→EtOAc/MeOH=9:1) to afford diol 5 (166 mg, 89%) as a colorless syrup. 1H NMR (300 MHz, CDCl₃): 1.79 (m, 1H, CH); 3.32 (m, 2H, CH₂N); 3.40 (bs, 2H, 2×OH); 3.63 (m, 4H, 2×CH₂OH); 5.08 (s, 2H, CH₂Ph); 5.45 (m, 1H, NH); 7.26-7.34 (m, 5H,Ph). 13C NMR (CDCl₃): 39.895, 43.747, 62.730, 67.408, 128.446, 128.853, 136.851, 158.153.

Synthesis of Di-Acetate 6

Diol 5 (82 mg, 0.346 mmol) is dissolved in CH₂Cl₂ (4 mL), then pyridine (0.2 mL) is added, followed by acetic anhydride (0.2 mL) and DMAP (9.4 mg). The mixture is stirred at room temperature for 14 h, then MeOH is added (0.1 mL), followed by H₂O (4 mL). The layers are separated, and the aqueous layer is extracted with EtOAc. The combined organic layers are dried (MgSO₄), filtered, concentrated, and purified by flash column chromatography (silica; Hexanes/EtOAc=1:1) to afford diacetate 6 (110 mg, 98%) as a colorless oil. ¹H NMR (300 MHz, CDCl₃): 2.05 (s, 6H, 2×COMe); 2.24 (m, 1H, CH); 3.26 (t, 2H, CH₂N); 4.11 (m, 4H, 2×CH₂OAc); 5.09 (s, 2H, CH₂Ph); 5.18 (m, 1H, NH); 7.30-7.36 (m, 5H, Ph). ¹³C NMR (CDCl₃): 20.909, 20.966, 38.908, 40.245, 62.921, 67.212, 128.394, 128.811, 136.928, 156.790, 171.052.

Procedure for the Preparation of 7 and ent-7

(a) 7 from enantioselective acetylation of diol 5: To a 100 mL jacketed flask equipped with a magnetic stir bar is added the diol 5 (10 g, 41.8 mmol, 1.00 eq) and tBuOH (32 mL). The resulting viscous solution was stirred at room temperature for 1 min. Then, vinyl acetate (3.59 g, 41.8 mmol, 1.00 eq) is added to the reaction mixture followed by 1.26 g of Pseudomonas cepacia lipase (commercially available as Amano PS from Amano, Nagbya-Japan). The suspension was then stirred at the same temperature for 90 min. Both the conversion and ee's of the monoacetate 7 is monitored by RP-HPLC. The reaction is stopped after the consumption of 70% of the starting material. Monoacetate 7 was produced with 97.5% ee. The heterogeneous mixture is filtered, and the solvent is removed in vacuo. Unconsumed diol 5 and monoacetate 7 are separated and purified by column chromatography (silica; Hexanes/EtOAc=1:2).

(b) ent-7 from enantioselective deacetylation of diacetate 6: Diacetate 6 (15 mg) is added to K₃PO₄ buffer (1 mL, 100 mM, pH 7.2) in a 8 ml glass vial containing a magnetic stir bar. The resulting solution is stirred at room temperature for 1 min. Then, 15 mg of Pseudomonas cepacia lipase (commercially available as Amano PS from Amano, Nagoya-Japan) is added, and the suspension is then stirred at the same temperature for 2 h. Both conversion and ee of monoacetate ent-7 are monitored by RP-HPLC. The reaction is stopped after the consumption of 30% starting material, which afforded monoacetate ent-7 with 97.5% ee. The heterogeneous mixture is filtered, and the solvent is removed in vacuo. Unconsumed diacetate 6 and monoacetate ent-7 are separated and purified by column chromatography (silica; Hexanes/EtOAc=1:2). ¹H NMR (300 MHz, CDCl₃): 1.96-2.06 (m, 4H, COCH₃, CH); 3.14 (bs, 1H, OH); 3.24 (m, 1H), 3.37 (m, 1H); 3.49-3.64 (m, 2H, CH₂N); 4.02-4.16 (m, 2H, CH₂OAc); 5.10 (s, 2H, CH₂Ph); 5.26 (m, 1H, NH); 7.31-7.36 (m, 5H, Ph). ¹³C NMR (CDCl₃): 20.997, 21.056, 39.743, 41.744, 60.906, 63.250, 67.388, 128.449, 128.885, 136.839, 157.744, 171.481.

Chiral-phase HPLC conditions: λ=254 nm; Chiralpak ADR-H, 3μ, C18, 4.6×150 mm; flow rate 0.5 mL/min; injection volume: 10 μL; mobile phases: A: water B: Acetonitrile; isocratic: 30% B for 20 min. Every HPLC sample was made by taking 2×20 μL from the reaction slurry, then combined and diluted with 4 ml of acetonitrile and injected in the HPLC.

Procedure for Enzyme Screening

The enzymatic screening for the desymmetrization of diol 5 was carried out as follows. A 96-well plate screening kit containing most of the commercially available hydrolases (lipases, proteases and esterases; plate was prepared in house) was used. The plate was lyophilized for 10 to 24 hours at a chamber temperature of −20° C. 100 μL of the substrate stock solution (50 mg of 1/mL vinyl acetate) was then added to each well via a multichannel pipette, and the 96 reactions were incubated at 30° C. and 750 rpm. The reactions were sampled after 1 hour and 16 hours by the transfer of 25 μL of the reaction mixture into a new 96-well plate, which then was quenched by the addition of 150 μL of acetonitrile. The 96-well plate was then centrifuged, and the organic supernatant transferred from each well into another 96-well plate. Sampled reactions were then sealed using a penetrable mat cover and transferred to an HPLC system for analysis. The same plate was used to analyze the samples for both reactivity and enantioselectivity using alternating columns on the HPLC simultaneously. Pseudomonas cepacia lipase was found to perform the reaction with >95% enantioselectivity and good reactivities, whereas Candida antarctica lipase B (CAL-B) and porcine pancreatic lipase (PPL) were found to provide the desired monoacetate 7 with lower degrees of enantioselectivity and reactivity.

Synthesis of Mono-Silylated (±)-8

(a) From Diol 5: To a solution of diol 5 (222 mg; 0.929 mmol) in dichloromethane (10 mL) is added imidazole (32 mg; 0.470 mmol) followed by tert-butyldimethylsilyl chloride (70 mg; 0.464 mmol) at room temperature. After stirring for 3 h at room temperature, another portion of imidazole (32 mg; 0.470 mmol) and tert-butyidimethylsilyl chloride (70 mg; 0.464 mmol) is added and the mixture is stirred at room temperature. After 16 h, a third portion of imidazole (32 mg; 0.470 mmol) and tert-butyidimethylsilyl chloride (70 mg; 0.464 mmol) is added and the mixture is stirred for another 3 h at room temperature. Then, half-saturated brine is added and the mixture is extracted with EtOAc. The combined organic layers are dried (MgSO₄), filtered, concentrated, and purified by flash column chromatography (silica; Hexanes/EtOAc=12:1) to afford racemic monosilylated product 8 (166 mg; 47%) as a colorless syrup.

(b) From Mono-Acetate (±)-7: To a solution of mono-acetate (±)-7 (2.12 g, 7.536 mmol) in CH₂Cl₂ (25 mL) is added imidazole (615 mg, 9.034 mmol), followed by TBSCI (1.36 g, 9.023 mmol). After stirring at room temperature for 2 h, aqueous saturated sodium bicarbonate solution is added and the layers are separated. The organic layer is washed once with aqueous saturated sodium bicarbonate solution. The combined aqueous layers are extracted once with EtOAc. The combined organic phases are dried (MgSO₄), filtered, concentrated, and purified by flash column chromatography (silica; Hexanes/EtOAc=2:1) to afford pure silylated mono-acetate, which is directly subjected to deacetylabon by dissolving in MeOH, addition of NaOMe and stirring for 2 h at room temperature. At this point, the deacetylation is complete. The mixture is quenched by addition of aqueous NH₄Cl-solution, extraction with EtOAc (4×), and drying of the combined organic phases (MgSO₄). Filtration, concentration, and purification of the residue by flash column chromatography (silica; Hexanes/EtOAc=12:1) afforded monosilylated product (±)-8 (2.55 g, 96% over 2 steps) as a colorless syrup. 1H NMR (300 MHz, CDCl₃): 0.00 (s, 6H, SiMe₂); 0.84 (s, 9H, SitBu); 1.79 (m, 1H, CH); 3.18-3.32 (m, 3H); 3.52-3.62 (m, 4H); 5.03 (s, 2H, CH₂Ph); 7.21-7.27 (n, 5H, Ph). 13C NMR (CDCl₃): 18.486, 26.075, 26.146, 26.251, 26.339, 40.805, 43.719, 60.609, 62.344, 63.947, 64.622, 67.066, 128.307, 128.806, 137.132, 157.538.

Synthesis of Aldehyde (±)-9

To a solution of (±)-8 (475 mg, 1.343 mmol) in dichloromethane (25 mL) is added Dess-Martin periodinane (626 mg, 1.478 mmol) at room temperature. After stirring for 15 min, the reaction is quenched by the addition of half-saturated aqueous NaHCO₃ solution, followed by Na₂S₂O₄ (2.1 g) while stirring vigorously. After phase separation, the organic phase is washed with water, and the combined aqueous phases are extracted with EtOAc. Then the combined organic phases are dried (MgSO₄), filtered, concentrated, and purified by flash column chromatography (silica; Hexanes/EtOAc=2:1) to afford aldehyde (±)-9 (402 mg; 85%) as a colorless syrup. ¹H NMR (300 MHz, CDCl₃): 0.00 (s, 6H, SiMe₂); 0.81 (s, 9H, SitBu); 2.63 (m, 1H, CH); 3.44-3.50 (m, 2H, CH₂N); 3.82 (dd, 1H, J=4.8 Hz, J=10.2 Hz, CH₂OSi); 4.00 (dd, 1H, J=4.8 Hz, J=10.5 Hz, CH₂OSi); 5.03 (s, 2H, CH₂Ph); 5.27 (m, 1H, NH); 7.21-7.29 (m, 5H, Ph); 9.71 (s, 1H, CHO). 13C NMR (CDCl₃): 18.462, 25.945, 26.035, 26.125, 26.212, 38.624, 54.576, 60.945, 67.103128.336, 128.775, 137.031, 156.779, 202.592.

Preparation of Ethyl 5-(bromomethyl)-4-metylthiophene-2-carboxylate 10

A solution of ethyl A-methyl-thiophene-2-carboxylate (12.16 g, 71.43 mmol) in CH₂Cl₂ (60 mL) is cooled in an icebath. To this solution are added slowly and carefully the following reagents in this order while maintaining the internal temperature below 5° C.: (a) 48% aqueous HBr-solution (54 mL); (b) 98% H₂SO₄ (27 mL); (c) ZnBr₂ (20.91 g); (d) 37% aqueous formaldehyde (9.2 mL). After the addition of formaldehyde is complete, the mixture is stirred for 16 h while warming to room temperature. Then, the two layers are separated, and the aqueous phase is extracted with CH₂Cl₂ (4×50 mL). The combined organic phases are washed with water (2×50 mL), saturated sodium bicarbonate solution (1×50 mL), and water (1×50 mL). Drying (MgSO₄), filtration, and removal of the solvent in vacuo afforded pure 10 as a solid (17.95 g, 95%). ¹H NMR (300 MHz, CDCl₃): 1.36 (t, J=5.1 Hz, 3H, CH₂CH₃); 2.24 (s, 3H, CH₃); 4.33 (quart, J=5.1 Hz, 2H, CH₂CH₃); 4.62(s, 2H, CH₂Br); 7.50 (s, 1H, CH). 13C NMR (CDCl₃): 13.827, 14.572, 24.506, 61.476, 136.171, 136.309, 138.232, 140.630, 162.187.

Ethyl 4-methyl-thiophene-2-carboxylate used in the above procedure is known in the art and may be prepared according to the methods described in (a) Oi, Satoru; Inatomi, Nobuhiro. PCT Int. Appl. (1998), WO 9842347 A1 19981001; (b) Marfat, Anthony; Robinson, Ralph P. U.S. (1998), Cont. of U.S. Ser. No. 960,208, abandoned U.S. Pat. No. 5,811,432 A 19980922; (c) Robinson, Ralph P.; Marfat, Anthony. Eur. Pat. Appl. (1991) EP 436333 A2 19910710; or (d) Gogte, V. N.; Tilak, Bal D.; Gadekar, Kumudini N.; Sahasrabudhe, M. B Tetrahedron 1967, 5, 2443-2451.

Compound of the formula 10 is known in the art and may alternatively be prepared according to the methods described in Choong, Ingrid; Burdeft, Matthew; Delano, Warren; Erianson, Daniel A.; Lee, Dennis; Lew, Willard. (Sunesis Pharmaceuticals, Inc., USA). PCT Int. Appl. (2003), PCT Int. Appl., WO03024955.

Preparation of Phosphonium Bromide 11

To a solution of 10 (5.75 g, 21.85 mmol) in toluene (60 mL) is added triethylphosphane (3.23 mL, 21.87 mmol), and the mixture is stirred at room temperature. After 1 min the mixture became turbid and then a very thick slurry. After stirring for 16 h at room temperature, the precipitate was filtered and washed with toluene/hexanes-mixtures. Drying in vacuum at 60° C. for 14 h afforded 11 (7.88 g, 94%) as a fine, white powder. ¹H NMR (300 MHz, CDCl₃): 1.22-1.41 (m, 12H, 4×CH₂CH₃); 2.44 (d, J=2.7 Hz, 3H, CH₃); 2.55-2.73 (m, 6H, 3×CH₂CH₃); 4.33 (quart, J=7.2 Hz, 2H, CH₂CH₃); 4.55 (d, 2H, J=75.3 Hz, CH₂P); 7.53 (s,1H, CH). 13C NMR (DMSO-d₆): 5.589, 5.660, 11.061, 11.689, 14.398, 14.537, 18.091, 19.524, 39.023, 39.302, 39.859, 40.137, 40.415, 40.693, 61.474, 131.739, 131.791, 131.957, 132.089, 136.709, 139.134, 139.228, 161.316

Coupling of (±)-9 and 11 via Wittig-Reaction—Formation of (±)-12

A suspension of Wittig-salt 11 (521.6 mg, 1.368 mmol) in anhydrous THF (6 mL) is cooled to ±65° C. and kept under argon. Then, NaH (55 mg, 1.375 mmol, 60% dispersion on mineral oil) is added, the cooling bath is removed, and the mixture is stirred for 60 min while warming to 18° C. To the yellow solution containing the Wittig-ylide, a solution of aldehyde (±)-9 (399 mg. 1.135 mmol) in THF (5 mL) is added. The color of the solution changed from yellow to orange. After stirring the reaction mixture for several hours at room temperature, half-saturated aqueous NH₄Cl solution (5 mL) is added, and the phases are separated. The aqueous phase is extracted with EtOAc (4×4 mL), and the combined organic phases are dried (MgSO₄), filtered, concentrated, and purified by flash column chromatography (silica; Hexanes/EtOAc=4:1) to afford the trans-olefine (±)-12 (461 mg; 75%) as a colorless syrup. 1H NMR (300 MHz, CDCl₃): 0.06 (s, 6H, SiMe₂); 0.89 (s, 9H, SitBu); 1.35 (t, J=7.2 Hz, 3H, CH₂CH₃); 2.18 (s, 3H, CH₃); 2.58 (m, 1H, CH); 3.32 (m, 1H, CH₂N); 3.46 (m, 1H, CH₂N); 3.65 (m, 1H, CH₂OTBS); 3.74 (m, 1H, CH₂OTBS); 4.31 (quart, 2H, J=7.2 Hz, CH₂CH₃); 5.09 (s, 2H, CH₂Ph); 5.19 (m, 1H, NH); 6.00 (dd, J=8.4 Hz, J=15.6 Hz, 1H, CH═CH); 6.59 (d, J=15.9 Hz, 1H, CH═CH); 7.26-7.32 (m, 5H, Ph); 7.46 (s, 1H, CH). 13C NMR (CDCl₃): −4.29, 13.356, 13.902, 15.586, 18.587, 25.353, 25.425, 27.01, 27.086, 61.393, 127.376, 127.790, 129.442, 129.915, 137.922, 156.813, 162.638.

Preparation of (±)-13—Desilylation of Wittig-Product (±)-12

A solution of (±)-12 (431 mg, 0.798 mmol) in THF (10 mL) is treated with a 1 M solution of TBAF in THF (0.85 mL) at room temperature for 30 min. Then, the mixture is concentrated, and the residue is purified by flash column chromatography (silica; Hexanes/EtOAc=1:1→1:2) to afford alcohol (±)-13 (308 mg; 96%) as a colorless syrup. 1H NMR (300 MHz, CDCl₃): 1.33 (t, J=7.2 Hz, 3H, CH₂CH₃); 2.17 (s, 3H, CH₃); 2.55 (m, 1H, CH); 3.24 (bs, 1H, OH); 3.31-3.45 (m, 2H); 3.57-3.71 (m, 3H); 4.29 (quart, J=7.2 Hz, 2H, CH₂CH₃); 5.10 (s, 2H, CH₂Ph); 5.34 (m, 1H, NH); 6.01 (dd, J=8.4 Hz, J=15.9 Hz, 1H, CH═CH); 6.59 (d, J=15.9 Hz, 1H, CH═CH); 7.26-7.33 (m, 5H, Ph); 7.45 (s, 1H, CH). ¹³C NMR (CDCl₃): 14.423, 14.824, 14.946, 42.371, 46.634, 61.729, 63.407, 67.686, 124.530, 128.737, 128.860, 129.189, 130.174, 131.204, 135.806, 136.948, 137.080, 137.117, 143.353, 158.140, 162.931.

Preparation of Malonate (±)-15 via Mesylate (±)-14

To a solution of alcohol (±)-13 (812.5 mg, 2.014 mmol) in CH₂Cl₂ (10 mL) is added triethylamine (365 μL, 2.619 mmol), followed by methanesulfonyl chloride (187 μL, 2.416 mmol). After stirring at room temperature for 2 h, the reaction mixture is filtered without work-up through flash column chromatography (silica; Hexanes/EtOAc=1:1) to afford mesylate (±)-14 (970 mg; 99%) as a colorless syrup after drying in vacuo. This material was used in the next step without further characterization.

A solution of diethylmalonate (810 μL, 5.335 mmol) in THF (4 mL) is treated with NaH (204 mg, 5.1 mmol, 60% dispersion on mineral oil) for 15 min at room temperature. This solution containing the malonate anion is transferred via syringe to a solution of mesylate (±)-14 (1.025 g, 2.128 mmol) in THF (6 mL) containing sodium iodide (80 mg, 0.534 mmol). This mixture is placed in an oilbath at 80° C. and heated for 8 h at this temperature. The mixture is quenched by addition of half-saturated aqueous NH₄Cl-solution, followed by EtOAc. The layers are separated and the aqueous phase is extracted with EtOAc. The combined organic phases are dried (MgSO₄), filtered, concentrated, and purified by flash column chromatography (silica; Hexanes/EtOAc=2:1) to afford malonate (±)-15 (711.3 mg; 61%) as a colorless syrup. 1H NMR (300 MHz, CDCl₃): 1.19-1.28 (m, 6H, 2×CH₂CH₃); 1.36 (t, J=7.2 Hz, 3H, CH₂CH₃); 1.90 (m, 1H); 2.11-2.20 (m, 4H); 2.42 (m, 1H); 3.17 (m, 1H); 3.29-3.42 (m, 2H); 4.03 (m, 4H, 2×CH₂CH₃); 4.32 (quart, J=7.2 Hz, 2H, CH₂CH₃); 4.90 (m, 1H, NH); 5.08 (s, 2H, CH₂Ph); 5.79 (dd, J=8.4 Hz, J=15.9 Hz, 1H, CH═CH); 6.53 (d, J=15.9 Hz, 1H, CH═CH); 7.32 (m, 5H, Ph); 7.45 (s, 1H, CH). 13C NMR (CDC): 13.742, 13.928, 14.036, 14.273, 18.582, 30.390, 37.187, 42.707, 45.006, 49.899, 61.037, 61.500, 61.578, 66.737124.691, 128.082, 128.465, 129.767. 131.785, 135.291, 136.389, 142.080, 156.245, 162.119, 169.010, 169.169.

Preparation of (±)-16 by Hydrogenation of (±)-15

To a solution of (±)-15 (102.3 mg, 0.187 mmol) in MeOH (6 mL) is added Pd(OH)₂/C (13.6 mg, 20 wt % Pd, wet), and the mixture is degassed and flushed with hydrogen several times. Then the mixture is stirred at room temperature under an atmosphere of hydrogen (1 atm). After stirring for 16 h under these conditions, monitoring by HPLC revealed complete consumption of starting material. The main product formed was (±)-16 together with a small amount of (±)-17 and 18 (vide infra). Filtration of the reaction mixture through Celite, concentration of the filtrate, and purification of the residue by flash column chromatography (silica; Hexanes/EtOAc=2:1) afforded (±)-16 (92 mg, 89%). A mixture of the free base of (±)-17 and 18 could be recovered by further elution of the column with neat EtOAc. 1H NMR (300 MHz, CDCl₃): 1.25 (m, 6H, 2×CH₂CH₃); 1.34 (t, J=7.2 Hz, 3H, CH₂CH₃); 1.63 (m, 3H); 1.94 (m, 2H); 2.13 (s, 3H, CH₃); 2.79 (m, 2H); 3.22 (m, 2H); 3.46 (m, 1H); 4.104.22 (m, 4H, 2×CH₂CH₃); 4.32 (quart, J=7.2 Hz, 2H, CH₂CH₃); 5.06-5.10 (m, 3H, NH, CH₂Ph); 7.27-7.35 (m, 5H, Ph); 7.45 (s, 1H, CH). 13C NMR (CDCl₃): 13.960, 14.426, 14.755, 25.943, 30.947, 33.258, 36.840, 43.552, 49.990, 61.228, 62.042, 67.134, 128.471, 128.908, 129.392, 134.506, 136.573, 136.611, 136.959, 146.815, 157.064, 162.741, 169.827.

Cbz-Cleavage and Lactamization—Preparation of Key Intermediate (±)-18

A solution of (±)-16 (107 mg. 0.195 mmol) in 33% HBr/AcOH (5 mL) is stirred at room temperature for 15 h after which monitoring by HPLC indicates complete consumption of starting material. Then EtOAc (20 mL) is added, followed by careful addition of saturated aqueous NaHCO₃-solution (60 mL) until the aqueous phase has pH=5, and, upon vigorous stirring, by addition of solid K₂CO₃ until pH=9. LC/MS indicates the presence of (±)-17 and 18. After phase separation has occurred, the organic phase is extracted with water and the combined aqueous phases are extracted with CH₂Cl₂, dried (MgSO₄), filtered, and concentrated to ˜25 mL volume. This mixture is treated with 5% aqueous KOH-solution (1 mL) and then warmed to 40° C. for 60 min. At this point, HPLC reveals 18 to be the only product present in the reaction mixture. Next, excess DABCO is added, and the mixture then is acidified with 3N HCl. Phase separation, washing of the organic phase with 3N HCl, followed by water, drying (MgSO₄), filtration, concentration, and purification of the residue by flash column chromatography (silica; EtOAc/MeOH mixtures) afforded 18 (56 mg, 77%) as a mixture of diastereomers. 1H NMR (300 MHz, CDCl₃): 1.25-1.49 (m, 6H, 2×CH₂CH₃); 1.64-2.26 (m, 5H); 2.78 (m, 2H, CH₂N); 3.03 (m, 1H, CH) 3.32-3.46 (m, 2H); 4.184.35 (m, 4H, 2×CH₂CH₃); 6.90 (bs, 1H, NH); 7.49 (s, 1H). 13C NMR (CDCl₃): 11.352, 13.998, 14.438, 14.498, 14.744, 23.369, 24.240, 26.003, 26.116, 29.315, 30.079, 30.753, 31.382, 32.554, 34.734, 39.125, 47.761, 49.230, 61.302, 61.832, 68.538, 129.190, 129.636, 129.690, 131.279, 132.841, 134.538, 136.605, 145.872, 146.019, 162.644, 168.141, 168.625, 170.873, 171.094.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

We claim:

1. A method of preparing a compound or a salt of formula (I): 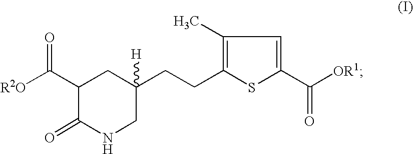
wherein each of R¹ and R² are independently a moiety that together with the attached CO₂ forms a readily hydrolyzable ester group;
wherein the method comprises the following steps:
(a) reacting a compound of formula (VI) selected from the group consisting of: 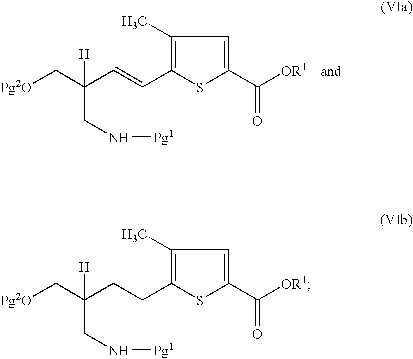
wherein R¹ is as described above, Pg¹ is an amino protecting group, and Pg² is an ether protecting group; with a deprotecting group in a solvent, to form a compound of formula (V) selected from the group consisting of: 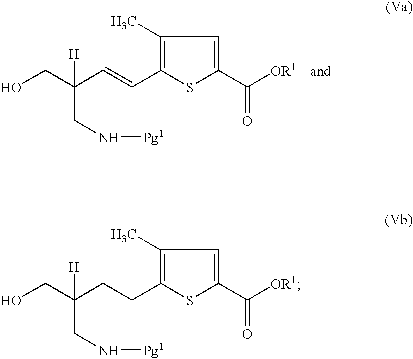
wherein each of Pg¹ and R¹ are as described above;
(b) reacting said compound of formula (V) with a leaving group producing agent in the presence of a base in a solvent, to form a compound of formula (IV) selected from the group consisting of: 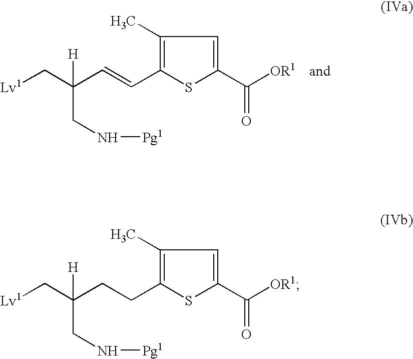
wherein each of Pg¹ and R¹ are as described above, and Lv¹ is a leaving group;
(c) reacting said compound of formula (IV) with a compound of formula (III): 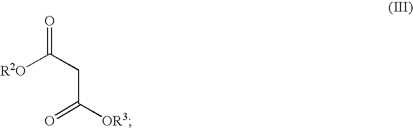
wherein R² is as described above, and R³ is a moiety that together with the attached CO₂ forms a readily hydrolyzable ester group; in the presence of a base in a solvent, to form a compound of formula (II) selected from the group consisting of: 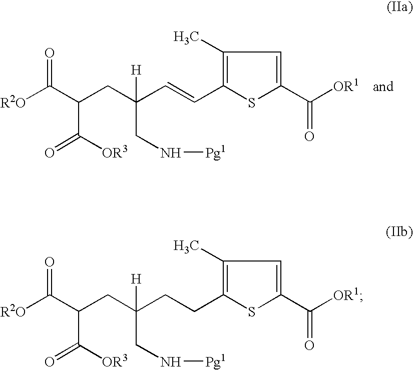
wherein each of Pg¹, R¹, R², and R³ are as described above; and
(d) reacting said compound of formula (II) with a cyclization reagent to form said compound of formula (I);
wherein the following hydrogenation step (e) is performed before any one of the above step (a), step (b), step (c), or step (d):
(e) hydrogenating the double bond in said compound (VIa), (Va), (IVa), or (IIa) by reacting said compound of formula (VIa), (Va), (IVa), or (IIa) with a hydrogenating agent in the presence of a catalyst and a solvent, to form a compound of formula (VIb), (Vb), (IVb), or (IIb); respectively.

2. A method according to claim 1, wherein the hydrogenation step (e) is perfomed before the above step (a) in the following manner:
(e) hydrogenating the double bond in compound (VIa) by reacting said compound of formula (VIa) with a hydrogenating agent in the presence of a catalyst and a solvent, to form a compound of formula (VIb):
(a) reacting said compound of formula (VIb) with a deprotecting group in a solvent, to form said compound of formula (Vb);
(b) reacting said compound of formula (Vb) with a leaving group producing agent in the presence of a base in a solvent, to form said compound of formula (IVb);
(c) reacting said compound of formula (IVb) with said compound of formula (III) in the presence of a base in a solvent, to form said compound of formula (IIb); and
(d) reacting said compound of formula (IIb) with a cyclization reagent to form said compound of formula (I).

3. A method according to claim 1, wherein the hydrogenation step (e) is perfomed before the above step (b) in the following manner:
(a) reacting said compound of formula (VIa) with a deprotecting group in a solvent, to form said compound of formula (Va);
(e) hydrogenating the double bond in compound (Va) by reacting said compound of formula (Va) with a hydrogenating agent in the presence of a catalyst and a solvent, to form said compound of formula (Vb);
(b) reacting said compound of formula (Vb) with a leaving group producing agent in the presence of a base in a solvent, to form said compound of formula (IVb);
(c) reacting said compound of formula (IVb) with said compound of formula (III) in the presence of a base in a solvent, to form said compound of formula (IIb); and
(d) reacting said compound of formula (IIb) with a cyclization reagent to form said compound of formula (I).

4. A method according to claim 1, wherein the hydrogenation step (e) is perfomed before the above step (c) in the following manner:
(a) reacting said compound of formula (VIa) with a deprotecting group in a solvent, to form said compound of formula (Va);
(b) reacting said compound of formula (Va) with a leaving group producing agent in the presence of a base in a solvent, to form said compound of formula (IVa);
(f) hydrogenating the double bond in said compound (IVa) with a hydrogenating agent in the presence of a catalyst and a solvent, to form said compound of formula (IVb);
(c) reacting said compound of formula (IVb) with said compound of formula (III) in the presence of a base in a solvent, to form said compound of formula (IIb); and
(d) reacting said compound of formula (IIb) with a cyclization reagent to form said compound of formula (I).

5. A method according to claim 1, wherein the hydrogenation step (e) is perfomed before the above step (d) in the following manner:
(a) reacting said compound of formula (VIa) with a deprotecting group in a solvent, to form said compound of formula (Va);
(b) reacting said compound of formula (Va) with a leaving group producing agent in the presence of a base in a solvent, to form said compound of formula (IVa);
(c) reacting said compound of formula (IVa) with said compound of formula (III) in the presence of a base in a solvent, to form said compound of formula (IIa);
(e) hydrogenating the double bond in said compound (IIa) by reacting said compound of formula (IIa) with a hydrogenating agent in the presence of a catalyst and a solvent, to form said compound of formula (IIb); and
(d) reacting said compound of formula (IIb) with a cyclizabon reagent to form said compound of formula (I).

6. A method according to claim 5, wherein in said step (d), said cyclization reagent is an acid in a solvent followed by a basic work-up.

7. A method according to claim 5, wherein in said step (d), the following intermediate of formula (VII) is formed: 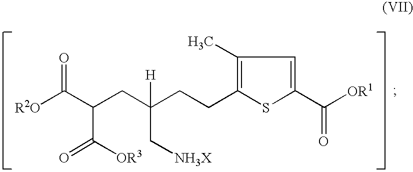
wherein R¹, R², and R³ are as described above, and X is halo.

8. A method according to claim 1, wherein each R¹, R², and R³ are independently C₁-C₆ alkyl or benzyl.

9. A method according to claim 1, wherein the method further comprises the following step of preparing said compound of formula (VIa):
(f-1) reacting a compound of formula (VIIIa): 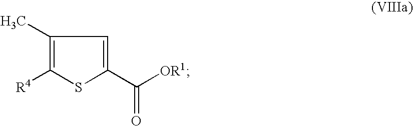
wherein R¹ is as described above and R⁴ is selected from the group consisting of —(CH₂PO(OR⁶)₂), —(CH₂P(R⁷)₃)⁺X⁻, —CH═PR⁷, —CH₂MX, —CH₂Si(R⁷)₃, and —CH₂SO₂(R⁷);
with a compound of formula (IXa): 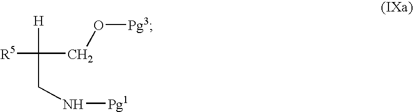
wherein Pg¹ is as described above, Pg³ is an ether protecting group, and R⁵ is —(C═O)—H; in the presence of a base, to form said compound of formula (VI).

10. A method according to claim 1, wherein the method further comprises the following steps of preparing said compound of formula (VIa):
(f-2) reacting a compound of formula (VIIIb): 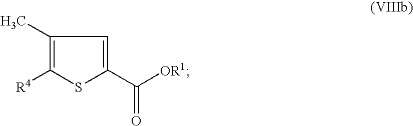
wherein R¹ is as described above and R⁴ is —C═O)—H;
with a compound of formula (IXb): 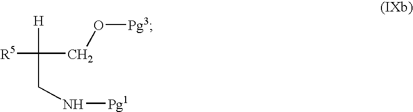
wherein Pg¹ is as described above, Pg³ is an ether protecting group, and R⁵ is selected from the group consisting of —(CH₂PO(OR⁶)₂), —(CH₂P(R⁷)₃)⁺X⁻, —CH═P(R⁷)₃, —CH₂MX, —CH₂Si(R⁷)₃, and —CH₂SO₂(R⁷); wherein each R⁶ and R⁷ is independently C₁-C₆ alkyl, —(CR¹³R¹⁴)ₜ(C₆-C₁₀ aryl) and —(CR¹³R¹⁴)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl; X is halo; and M is a metal; in the presence of a base in an inert solvent, to form said compound of formula (VI).

11. A method according to claim 9, wherein said R⁴ is —(CH₂P(R⁷)₃)⁺X⁻, wherein R⁷ is C₁-C₆ alkyl, or benzyl.

12. A method according to claim 9, wherein the method further comprises the following steps of preparing said compound of formula (VIIIa):
(g) reacting a compound of formula (X): 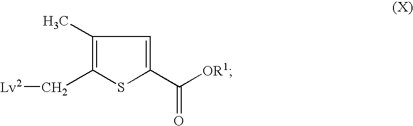
with an activating agent wherein R¹ is as described above and Lv² is a leaving group, in a solvent, to form said compound of formula (VIIIa).

13. A method according to claim 9, wherein the method further comprises the following steps of preparing said compound of formula (IXa):
(h) reacting a compound of formula (XI): 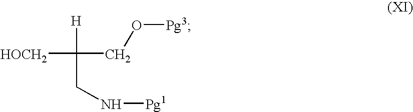
with an oxidizing agent in a solvent, to form said compound of formula (IXa).

14. A method according to claim 9, wherein the method further comprises the following steps of preparing said compound of formula (XI):
(i-1) reacting a compound of formula (XII): 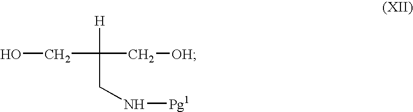
wherein Pg¹ is as described above;
with a compound of formula R⁹—(O═C)—O—(C═O)—R⁹; wherein R⁹ is selected from the group consisting of C₁-C₆ alkyl, —(CR¹³R¹⁴)ₜ(C₆-C₁₀ aryl) and —(CR¹³R¹⁴)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl, to form a compound of formula (XIII): 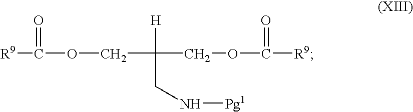
wherein Pg¹ and R⁹ are independently as described above;
(i-1) exposing said compound (XIII), to an enzyme, in the presence of a buffer, to form a compound of formula (XIV): 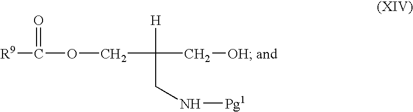
(j-2) reacting said compound of formula (XIV) with a Pg³ producing agent in the presence of a base, followed by base-induced cleavage of R⁸—(C═O)— to form said compound of formula (XI).

15. A method according to claim 14, wherein in the step (j-1), the method is stereoselective and provides an enantiomeric excess of at least 60% to 99.9%.

16. A method according to claim 9, wherein the method further comprises the following steps of preparing said compound of formula (XI):
(i-2) exposing a compound of formula (XII): 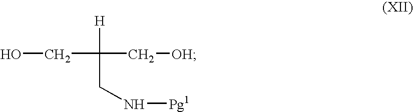
wherein Pg¹ is as described above;
to an enzyme, in the presence of a compound of formula CH₂═CH—O—(C═O)—R⁸, to form a compound of formula (XIV): 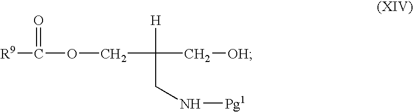
(j-2) reacting said compound of formula (XIV) with a Pg³ producing agent in the presence of a base, followed by base-induced cleavage of R⁸—(C═O)— to form said compound of formula (XI).

17. A method according to claim 16, wherein in step (i-2), the method is stereoselective and provides an enantiomeric excess of at least 60% to 99.9%.

18. A method according to claim 9, wherein the method further comprises the following steps of preparing said compound of formula (XI):
(i-3) reacting a compound of formula (XII): 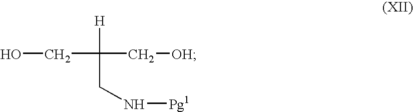
wherein Pg¹ is as described above;
with a Pg³ producing agent in the presence of a base, to form said compound of formula (XI).

19. A method according to claim 14, wherein said R⁹ is methyl and said enzyme is P. cepacia lipase.

20. A method according to claim 16, wherein each of said R⁸ is CH₃, said enzyme is P. cepacia lipase, and said step (i-2) is performed in the presence of a compound of formula CH₂═CH—O—(C═O)—CH₃ in an alcoholic solvent.

21. A method according to claim 14, wherein the method further comprises the following steps of preparing said compound of formula (XII):
(k) reacting a compound of formula (XV): 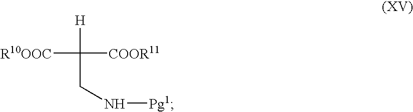
wherein each of R¹⁰ and R¹¹ are independently a moiety that together with the attached CO₂ forms a readily hydrolyzable ester group;
with a reducing agent, to form said compound of formula (XII).

22. A method according to claim 21, wherein the method further comprises the following steps of preparing said compound of formula (XV):
(o) reacting a compound of formula (XVIII):
R¹⁵—O—(C═O)—NH₂ (XVIII);
wherein R¹⁵ is C₁-C₆ alkyl, —(CR¹³R¹⁴)ₜ(C₆-C₁₀ aryl) and —(CR¹³R¹⁴)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl;
with formaldehyde in the presence of a base and a solvent, to form a compound of formula (XVII): 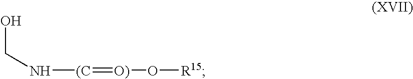
wherein R¹⁵ is as described above;
(p) reacting said compound of formula (XVII) with a compound of formula R¹²—(O═C)—O—(C═O)—R¹², wherein R¹² is C₁-C₆ alkyl, —(CR¹³R¹⁴)ₜ(C₆-C₁₀ aryl) and —(CR¹³R¹⁴)ₜ(4-10 membered heterocyclic), wherein t is an integer from 0 to 5; 1 or 2 ring carbon atoms of the heterocyclic group are optionally substituted with an oxo (═O) moiety; and each R¹³ and R¹⁴ is independently H or C₁-C₆ alkyl; to form a compound of formula (XVI):
Pg¹—NH—CH₂—O—(C═O)—R¹² (XVI);
wherein Pg¹ and R¹² are independently as described above;
(q) reacting said compound of formula (XVI) with a compound of formula R¹¹—O—(O═C)—CH₂—(C═O)—O—R¹⁰, wherein each of R¹⁰ and R¹¹ are independently a moiety that together with the attached CO₂ forms a readily hydrolyzable ester group, in the presence of a base and solvent to form said compound of formula (XV).

23. A compound which is:

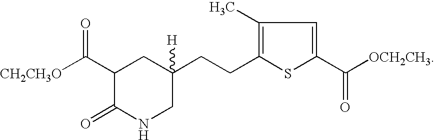

24. A compound of formula (V): 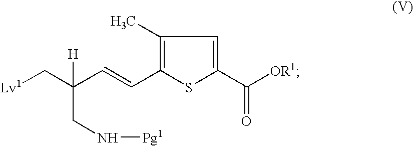
wherein Pg¹ is an amino protecting group, R¹ is a moiety that together with the attached CO₂ forms a readily hydrolyzable ester group, and Lv¹ is a leaving group.

25. A method according to claim 16, wherein the method further comprises the following steps of preparing said compound of formula (XII):
(k) reacting a compound of formula (XV): 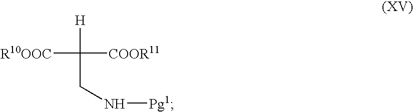
wherein each of R¹⁰ and R¹¹ are independently a moiety that together with the attached CO₂ forms a readily hydrolyzable ester group;
with a reducing agent, to form said compound of formula (XII).
